(19) United States
(12) Patent Application Publication
(10) Pub. No.: US 2008/0135043 A1
(43) Pub. Date: Jun. 12, 2008

(54) Inhalation Devices

(75) Inventors: Richard David Lintern, Cambridgeshire (GB); Allen John Pearson, Cambridgeshire (GB); Paul Kenneth Rand, Hertfordshire (GB)

Correspondence Address: GLAXOSMITHKLINE;CORPORATE INTELLECTUAL PROPERTY, MAI B475, FIVE MOORE DR., PO BOX 13398, RESEARCH TRIANGLE PARK, NC, 27709-3398, US

(73) Assignee: Glaxo Group Limited

(21) Appl. No.: 11/908,508

(22) PCT Filed: Mar. 16, 2006
(86) PCT No.: PCT/GB2006/000963

§ 371 Date: Sep. 13, 2007

(30) Foreign Application Priority Data
Mar. 17, 2005 (GB) 0505545.4

(51) Int. Cl. A61M 15/00

(52) U.S. Cl. 128/200.23

(57) ABSTRACT

An inhaler for delivering medicament by inhalation comprises a canister which comprises a body which includes a base and a head and defines a chamber containing medicament, and a valve stem which extends from the body and from which medicament is in use delivered on actuation of the canister. The inhaler also comprises an actuator comprising a main body comprising a housing receiving the canister, and an actuation mechanism for actuating the canister. The actuation mechanism comprises a loading member which is fitted to or comprised in the canister and includes a loading section which is located at a distance spaced from the base of the body of the canister and, in use, acted upon to drive the loading member in an actuating direction from a first, rest position to a second, actuated position in which the canister is actuated to deliver medicament. The actuation mechanism also comprises at least one actuating member which is actuatable by a user to drive the loading member in the actuating direction to the actuated position, such as to actuate the canister to deliver medicament. The at least one actuating member is pivotally coupled to the housing for pivotal movement relative to the housing from a first, rest position to a second, actuated position in which the loading member is driven in the actuating direction to the actuated position, such as to actuate the canister to deliver medicament. The at least one actuating member comprises a gripping element which extends along a length of the housing and is configured to be gripped and depressed by the user in actuating the canister.

### FIELD OF THE INVENTION

The present invention relates to an actuator for an inhaler for administering medicament by inhalation and to an inhaler including the same. The invention is particularly, but not exclusively, concerned with an actuator for a pressurised metered dose inhaler (pMDI).

### BACKGROUND OF THE INVENTION

pMDIs are well known in the art of inhalation devices. It is therefore not necessary to describe the construction and operation of a pMDI other than in bare essentials.

A pMDI comprises a canister and an actuator housing. The housing is generally tubular, although this is not essential, and generally formed of a plastics material, for instance by moulding. The canister comprises an open-ended canister, typically made from a metal such as aluminium. The open end of the canister is sealingly capped by a metering valve assembly. The valve assembly typically includes a hollow dispensing member or valve stem which projects from the outlet or business end of the canister. The dispensing member is mounted for sliding movement relative to the canister between an extended position, to which the dispensing member is biased by a biasing mechanism in the valve assembly, typically a return spring, and a depressed position.

In use, the sealed canister contains a pressurised medicinal aerosol formulation. The formulation comprises the medicament and a fluid propellant, and optionally one or more excipients and/or adjuvants. The medicament is typically in solution or suspension in the formulation. The propellant is typically a CFC-free propellant, suitably a liquid propellant, and may for example be HFA-134a or HFA-227.

Movement of the dispensing member from the extended position to the depressed position results in a metered dose of the aerosol formulation being dispensed from the canister through the dispensing member. Typically, the metering valve assembly is provided with a metering chamber of defined volume. In the extended position of the dispensing member, the content of the canister is placed in fluid communication with the metering chamber through the dispensing member so that the metering chamber is filled with the aerosol formulation. When the dispensing member is depressed, the metering chamber is isolated from the canister inner volume and placed in fluid communication with the external environment through the dispensing member. Thus, the defined volume of the aerosol formulation in the metering chamber is discharged to the external environment via the dispensing member.

Such metering valve assemblies are well known in the art and can be obtained from inter alia Bespak Plc (King's Lynn, Norfolk, United Kingdom) and Valois S.A.S. (Le Neubourg, France).

The housing typically comprises an internal passageway having an open end. The canister is slidable into the internal passageway through the open end with the canister being inserted valve assembly first into the internal passageway. A stem block, which receives the dispensing member of the canister when the canister is received in the housing in a “rest position”, has a passageway with an inlet end for receiving the dispensing member and an outlet end, which faces a dispensing outlet of the housing, typically a mouthpiece or a nasal nozzle. The stem block holds the dispensing member stationary whereby depression of the canister to its rest position further into the housing to an “actuated position” causes the dispensing member to be displaced from the extended position to the depressed position relative to the canister. A metered dose of the aerosol formulation will thereby be dispensed out of the dispensing outlet of the housing via the internal passageway of the stem block.

In use, a patient in need of a metered dose of the medicinal aerosol formulation concurrently inhales on the dispensing outlet and depresses the canister from the rest position to the actuated position. The inspiratory airflow produced by the patient entrains the metered dose of the medicinal aerosol formulation into the patient's respiratory tract. So, a pMDI of the type described above is a breath-coordinated inhaler.

Inhalers are commonly provided with a dust cap that covers the dispensing outlet when the inhaler is not in use. The dust cap, when applied, prevents foreign material from entering the housing. This prevents the user from inhaling dust or lint, for example, that might otherwise accumulate in the housing. This is of particular importance where the user suffers from asthma or other respiratory conditions, in which the inhalation of foreign material may cause severe irritation.

Developments to pMDIs have included the provision of actuation indicators or dose counters therefor. Such a dose counter is described in PCT Patent Application Nos. WO-A-9856444 (U.S. Pat. No. 6,431,168) and WO-A-2004/001664 (U.S. Ser. No. 10/518,421) to Glaxo Group Limited, the entire contents of each of these patent applications and patents hereby being incorporated herein by reference. The dose counter is fixably secured on the valve assembly end of the canister and includes a display which denotes the number of metered doses of the medicament formulation dispensed from, or remaining in, the canister. The dose counter is preferably permanently secured on the valve assembly end as described in US-A-2003/0136800 or WO-A-2004/065224 (U.S. Ser. No. 10/543,049), the entire contents of each of these patent applications hereby being incorporated herein by reference. The display of the dose counter is visible to the patient through a window provided in the housing. The display may be presented by a plurality of indicator wheels rotatably mounted on a common axle, each wheel having numerals displayed in series around the circumference.

Many actuators have been developed with a view to facilitating the delivery of medicament, examples of which are disclosed in U.S. Pat. No. 3,272,391, U.S. Pat. No. 3,272,392, U.S. Pat. No. 4,678,106, U.S. Pat. No. 5,899,365, U.S. Pat. No. 6,237,812 and WO-A-99/49917.

It is an aim of the present invention to provide an improved actuator for an inhaler for administering medicament by inhalation and an inhaler including the same.

### SUMMARY OF THE INVENTION

In one aspect the present invention provides an inhaler for delivering medicament by inhalation, comprising:

a canister which comprises a body which includes a base and a head and defines a chamber containing medicament, and a valve stem which extends from the body and from which medicament is in use delivered on actuation of the canister; and

an actuator comprising a main body comprising a housing receiving the canister, and an actuation mechanism for actuating the canister;

the actuation mechanism comprising a loading member which is fitted to or comprised in the canister and includes a loading section which is located at a distance spaced from the base of the body of the canister and, in use, acted upon to drive the loading member in an actuating direction from a first, rest position to a second, actuated position in which the canister is actuated to deliver medicament, and at least one actuating member which is actuatable by a user to drive the loading member in the actuating direction to the actuated position, such as to actuate the canister to deliver medicament;

the at least one actuating member being pivotally coupled to the housing for pivotal movement relative to the housing from a first, rest position to a second, actuated position in which the loading member is driven in the actuating direction to the actuated position, such as to actuate the canister to deliver medicament; and

the at least one actuating member comprising a gripping element which extends along a length of the housing and is configured to be gripped and depressed by the user in actuating the canister.

The loading member may be located over the base of the body of the canister.

The loading member may comprise a sleeve which fits about an outer peripheral surface of the body of the canister, an end section at one end of the sleeve which engages the base of the body of the canister, and the loading section at the other end of the sleeve.

The sleeve of the loading member may extend substantially to the head of the body of the canister.

The loading section of the loading member may comprise a substantially annular section.

The loading member may be attached substantially to the head of the body of the canister.

The housing may include at least one lateral opening in which the at least one actuating member is disposed for depression by the user.

The actuation mechanism may comprise first and second actuating members which are disposed in oppositely-directed relation. The actuation mechanism may further comprise at least one toggle element which acts normally to bias the loading member in a direction opposite the actuating direction and is configured to toggle between a first, restraining configuration and a second, actuated configuration on the application of a predeterminable actuation force to the loading member in the actuating direction.

The at least one toggle element may comprise an elongate resilient element which extends substantially orthogonally to the actuating direction and engages the loading member such as normally to adopt a first bowed configuration which biases the loading member in a direction opposite the actuating direction and, on application of the actuation force, is toggled to a second, oppositely bowed configuration, which allows for actuation of the canister.

The second bowed configuration may not be a stable state, such that, on release of the actuation force, the at least one toggle element returns to the first bowed configuration, thereby causing the loading member to return to the rest configuration.

The actuation mechanism may comprise first and second toggle elements which are disposed to opposed sides of the canister.

The main body may include a nozzle block which receives the valve stem of the canister.

The housing may include an outlet member through which the user in use inhales.

The outlet member may be a mouthpiece.

In an embodiment of the invention, the loading member is fitted to the head of the canister body, preferably permanently fitted to the head. The canister body may have an outer peripheral surface extending from the base to the head and the loading member may be provided with a sleeve which is disposed about the outer peripheral surface adjacent the head and which presents the loading section.

The loading member may be attached at a neck of the canister body. The loading member may comprise a sleeve having an inner surface in opposed relation to the neck and an annular element which is mounted on the neck and connected to the inner surface. The annular element may present the loading section.

In another aspect of the present invention there is provided the actuator of the inhaler of the invention.

Other aspects and features of the invention are set forth in the appended claims and the exemplary embodiments which will now be described with reference to the accompanying Figures of drawings.

### BRIEF DESCRIPTION OF THE DRAWINGS

FIG. 1 illustrates a perspective view of an inhaler in accordance with a first embodiment of the present invention;

FIG. 2 illustrates a part-sectional view of an inhaler corresponding closely to FIG. 1, where illustrated in the rest or inoperative configuration;

FIG. 3 illustrates a part-sectional view of the inhaler of FIG. 2, where illustrated in the actuated configuration;

FIG. 4 illustrates a perspective view of the actuation mechanism of an inhaler in accordance with a second embodiment of the present invention, where illustrated in the rest or inoperative configuration;

FIG. 5 illustrates a part-sectional view of the inhaler of FIG. 4, where illustrated in the actuated configuration;

FIG. 6 is a perspective front view of an inhaler in accordance with a third embodiment of the present invention;

FIG. 7 is another perspective front view of the inhaler of the third embodiment showing how an upper part of the actuator is separable from a lower, mouthpiece part of the actuator for access to the aerosol canister enclosed therewithin;

FIG. 8 is a further perspective front view of the inhaler of the third embodiment with the upper and lower actuator parts separated and the aerosol canister removed;

FIG. 9 is a part-sectional view of the inhaler of the third embodiment showing the inhaler in its rest or inoperative configuration;

FIG. 10 is a part-sectional view of the inhaler of the third embodiment showing the inhaler in its actuated configuration;

FIG. 11 is an exploded view of the upper actuator part of the inhaler of the third embodiment;

FIG. 12 is an exploded view of an alternative upper actuator part for the inhaler of the third embodiment;

FIG. 13 illustrates a perspective view of an inhaler in accordance with a fourth embodiment of the present invention;

FIGS. 14(a) and 14(b) are part-sectional views of an inhaler corresponding closely to FIG. 13, where illustrated respectively in the rest or inoperative configuration and in the actuated configuration;

FIG. 15 illustrates an aerosol canister assembled with a loading member in the inhaler of FIGS. 13 and 14;

FIG. 16 illustrates a part-sectional view of an inhaler in accordance with a fifth embodiment of the invention in which a dose counter is permanently fixed to an aerosol canister, where illustrated in the rest or inoperative configuration;

FIG. 17 illustrates a part-sectional view of the inhaler of FIG. 16, where illustrated in the actuated configuration;

FIGS. 18(a) to 18(c) are schematic illustrations of the process by which the aerosol canister and the dose counter in the inhaler of FIGS. 16 and 17 are permanently fixed together; and

FIGS. 19(a) to 19(c) correspond to FIGS. 18(a) to 18(c), respectively, but with the dose counter having a different configuration.

### DETAILED DESCRIPTION OF PREFERRED EMBODIMENTS

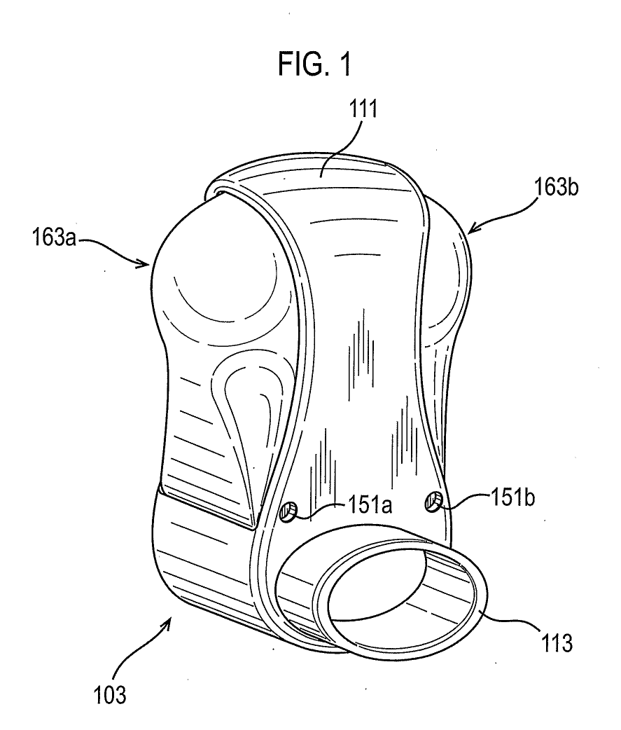

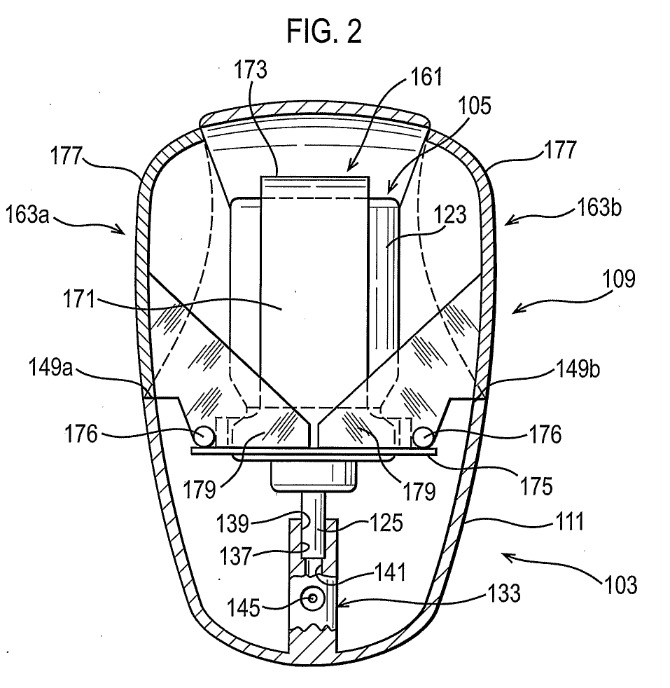

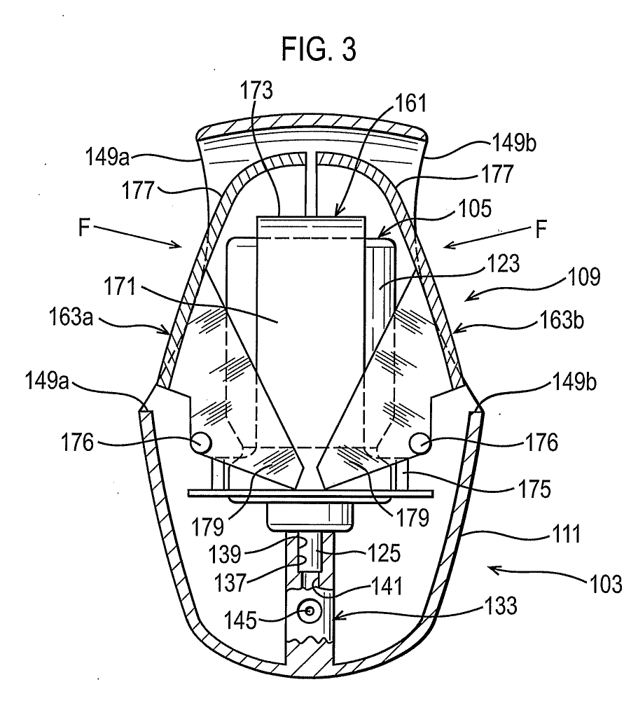

FIGS. 1 to 3 illustrate a hand-held, hand-operable inhaler of the pMDI type in accordance with a first embodiment of the present invention, noting that there are some styling differences between the representation in FIG. 1 and the representations in FIGS. 2 and 3.

The inhaler comprises an actuator which comprises a main body 103, an aerosol canister 105 which is fitted in the main body 103 and contains medicament to be delivered on actuation of the inhaler, and an actuation mechanism 109 which is operable by a user to actuate the inhaler.

The main body 103 comprises a housing 111 in which the canister 105 is in use fitted, and a mouthpiece 113, in this embodiment a tubular element, which is in fluid communication with one, the lower, end of the housing 111 and in use is gripped in the lips of the user. The mouthpiece 113 could instead be configured as a nasal nozzle.

The canister 105 in this embodiment is of standard type, as outlined supra, and comprises a body 123 which includes a base and a head and defines a chamber containing a medicament in a CFC-free propellant under pressure, for example an HFA propellant, a valve stem 125 which extends from the head of the body 123 and an internal metering valve (not illustrated) which is normally biased by an internal valve spring (not illustrated) to a closed position and opened to deliver a metered dose of medicament from the canister 105 when the valve stem 125 is depressed into the canister body 123.

The housing 111 includes a nozzle block 133, in this embodiment disposed to a base surface of the housing 111, for receiving the valve stem 125 of the canister 105.

Referring particularly to FIGS. 2 and 3, the nozzle block 133 includes a tubular bore 137 for receiving the valve stem 125 of the canister 105, which in this embodiment is co-axial with the longitudinal axis of the housing 111. The tubular bore 137 is open at one, the upper, end thereof and includes an upper section 139 which has an internal dimension which is substantially the same as the outer dimension of the valve stem 125 and a lower section 141 which has a smaller dimension, which sections 139, 141 together define an annular seat for the distal end of the valve stem 125. The tubular bore 137 further includes a laterally-directed spray orifice 145 in the lower section 141 thereof which is configured to direct a spray into and through the mouthpiece 113.

The housing 111 further includes first and second lateral apertures 149a, b, in this embodiment elongate apertures, which are disposed in opposed relation to lateral sides of the mouthpiece 113 and receive actuating members 163a, b of the actuation mechanism 109, as will be described in more detail hereinbelow. The actuating members 163a, b are configured and arranged in the apertures 149a, b such that a gap (not shown) is formed therebetween. The gap functions as an air inlet to the housing 111 in the sense that, when a patient inhales at the mouthpiece 113, air is drawn into the housing 111 through the gap and flows out of the mouthpiece 113 into the patient's respiratory tract. When such inhalation is coordinated with operation of the actuation mechanism 109 to release medicament from the canister 105, the medicament is entrained in this inhalation airflow for delivery to the patient's lungs (or nasal cavity if the mouthpiece 113 is configured as a nasal nozzle).

The housing 111 further includes first and second pivot elements 151a, b which are disposed at the respective lower ends of the lateral apertures 149a, b to which a respective one of the actuating members 163a, b is pivoted, as will be described in more detail hereinbelow. In this embodiment the pivot elements 151a, b each comprise a pair of pivot apertures.

In this embodiment the housing 111 is formed, here by moulding, as a single, integral unit.

The actuation mechanism 109 comprises a loading member 161 which is fitted over the base of the body 123 of the canister 105, and the first and second actuating members 163a, b which are disposed at the respective ones of the lateral apertures 149a, b in the housing 111 and pivotally mounted to the housing 111 for pivotal movement between a first, rest or inoperative configuration, as illustrated in FIG. 2, and a second, actuated configuration, as illustrated in FIG. 3, such as to provide for actuation of the canister 105 by engagement with the loading member 161.

In this embodiment the loading member 161 is slidably disposed relative to the nozzle block 133 between a first, rest or inoperative position, as illustrated in FIG. 2, and a second, actuated position in which the canister 105 is actuated, as illustrated in FIG. 3, and comprises a sleeve 171, here a tubular sleeve, which is a close fit with the outer peripheral wall of the body 123 of the canister 105, an end section 173 at one, the upper, end of the sleeve 171, here which spans the sleeve 171, which engages the base of the body 123 of the canister 105, and a loading section 175, here of annular form to present an annular flange, at the other, lower end of the sleeve 171, which is engaged by the actuating members 163a, b to load the canister 105, as will be described in more detail hereinbelow. The loading section 175 need not necessarily be annular, but instead provide first and second lateral flanges for the actuating members 163a, b to act on.

In this embodiment the actuating members 163a, b each include a pivot element 176 which engages the counterpart pivot element 15la, b in the housing 111, and further each comprise a first, gripping arm 177 which extends across the respective lateral aperture 149a, b in the housing 111 and is configured to be gripped and depressed by the user in actuating the inhaler (e.g. with opposing digits of a user's hand), and a pair of second, loading arms 179 (only one shown per actuating member 163a, b) which extend inwardly from the respective pivot element 176, in a direction substantially orthogonal to the gripping arm 177 so as to straddle the loading member 161, thus defining substantially an inwardly-directed L shape, and is operative to engage the loading section 175 of the loading member 161.

Operation of the actuator will now be described hereinbelow.

The user first takes the actuator in the rest or inoperative configuration, as illustrated in FIG. 2, in one hand.

The user then takes the mouthpiece 113 in his/her lips, and, in co-ordination with an inhalation breath, actuates the inhaler by depressing the gripping arms 177 of the actuating members 163a, b with one or more digits of the hand holding the actuator.

As illustrated in FIG. 3, depression of the gripping arms 177 of the actuating members 163a, b causes the inward rotation of the actuating members 163a, b, such that the loading arms 179 of the actuating members 163a, b drive the loading section 175 of the loading member 161, and hence the loading member 161, downwardly, which downward movement of the loading member 161 drives the canister body 123 of the canister 105 downwardly in relation to the valve stem 125 of the canister 105 which is held stationary by the nozzle block 133. More particularly, when the actuating members 163a, b are pivoted inwardly, the loading arms 179 operate on the loading section 175 to push the loading member 161 downwardly. This in turn causes the end section 173 of the loading member 161 to bear on the base of the canister body 123 and to push the canister body 123 downwardly in the housing relative to the stationary valve stem 125.

This downward movement of the canister body 123 in relation to the stationary valve stem 125 actuates the canister 105 to deliver a spray of the medicament formulation dispensed from the valve stem 125 into and through the mouthpiece 113.

On releasing the actuating members 163a, b, the inhaler is returned by the valve return spring to the rest configuration illustrated in FIG. 2, ready for subsequent actuation.

Following actuation, the inhaler is removed from the mouth, ready for subsequent actuation.

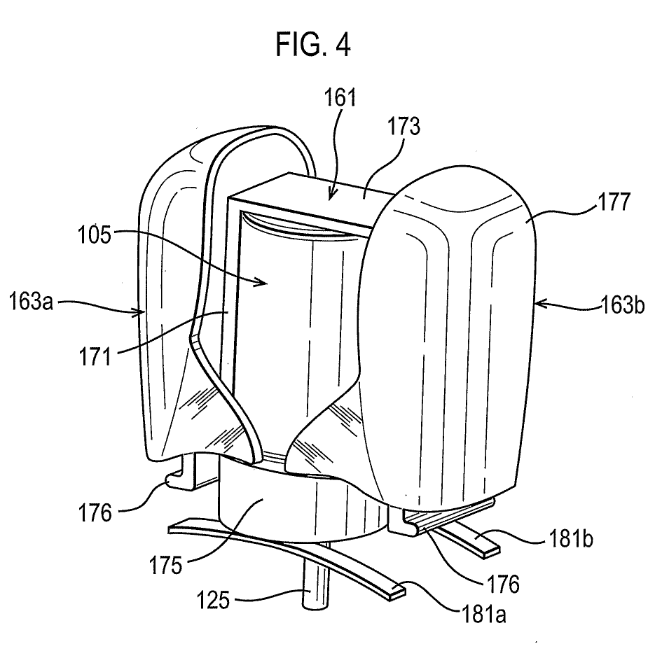

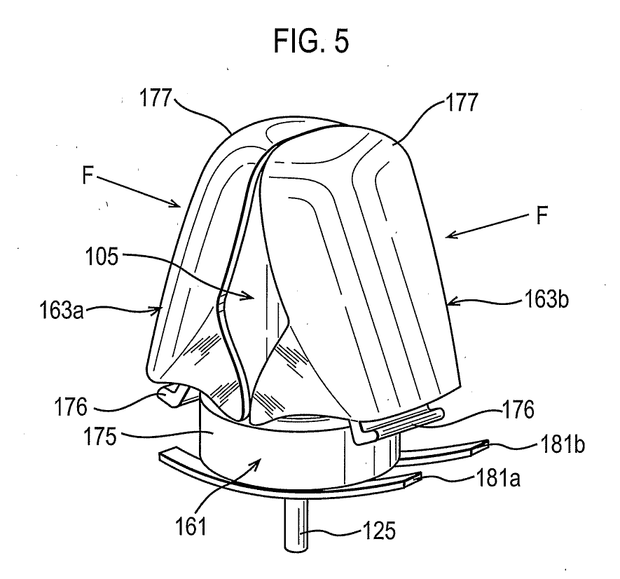

FIGS. 4 and 5 illustrate the actuation mechanism of a hand-held, hand-operable inhaler of the pMDI type in accordance with a second embodiment of the present invention.

The inhaler of this embodiment is very similar to the inhaler of the above-described embodiment, and thus, in order to avoid unnecessary duplication of description, only the differences will be described in detail, with like parts being designated by like reference signs.

The inhaler of this embodiment differs from that of the first-described embodiment in the construction of the actuation mechanism 109.

In this embodiment the pivot elements 151a, b in the housing 111 each comprise a recess, here in the form of a slot, and the pivot element 176 of each of the actuating members 163a, b comprises a flange which is pivotally engaged in a respective one of the pivot elements 151a, b in the housing 111.

In this embodiment the actuation mechanism 109 further comprises at least one toggle element 181, here first and second toggle elements 181a, b which act normally to bias the loading member 161 in a direction, here upwards, against the operative action of the actuating members 163a, b and are configured to toggle or switch between a first, restraining configuration, as illustrated in FIG. 4, and a second, actuated configuration, as illustrated in FIG. 5, on the application of a predetermined actuation force to the actuating members 163a, b by the user. With this configuration, instantaneous actuation of the inhaler at a predetermined actuation force, which is such as to cause the toggling of the at least one toggle element 181, is ensured. Such actuation provides for a uniform dose delivery with each operation, and prevents the possibility of a variable dose delivery over repeated operations, which could be achieved by slowly squeezing the actuating members 163a, b in the absence of the at least one toggle element 181.

The toggle element 181 can be considered as a ‘commitment’ feature in the sense that once the threshold (commitment) actuation force needed to toggle the toggle element 181 is applied by the user, there is an instantaneous actuation of the inhaler.

In this embodiment the at least one toggle element 181 comprises an elongate resilient element, typically formed of a spring metal, which extends substantially orthogonally to the direction of movement of the loading member 161 and engages the loading section 175 of the loading member 161 to the opposite side to the loading arms 179 of the actuating members 163a, b, such as normally to adopt a first bowed configuration which biases the loading member 161 upwardly and, on application of a predetermined actuation force, is toggled or switched to a second, oppositely bowed configuration, which allows for actuation of the canister 105. The actuation mechanism 109 is configured such that the second bowed configuration is not a stable state, such that, on release of the actuating members 163a, b by the user, the at least one toggle element 181 returns to the first bowed configuration, thereby causing the loading member 161, and hence the canister 105 and the actuating members 163a, b, to return to the rest or inoperative configuration.

Operation of the inhaler of this embodiment is otherwise the same as for the above-described first embodiment.

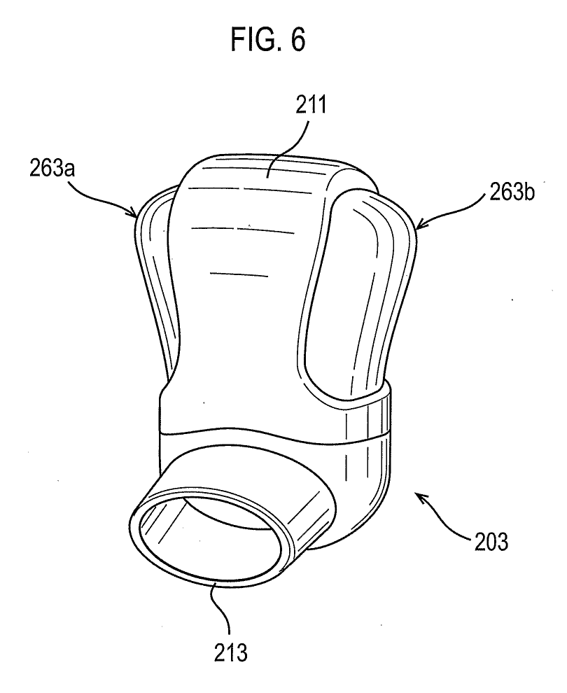

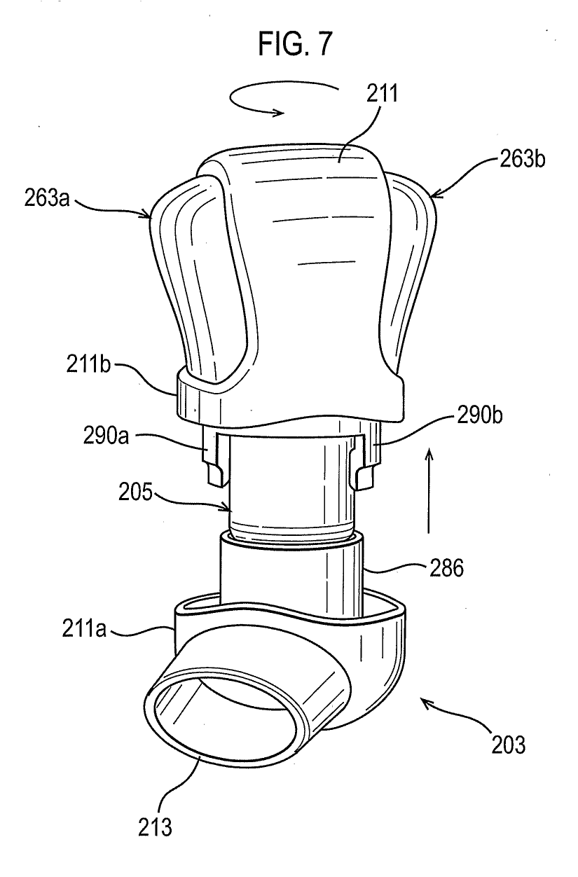

The canister 105 in the embodiments illustrated in FIGS. 1 to 5 may be furnished with a dose counter on its head as described in WO-A-9856444 (U.S. Pat. No. 6,431,168) and WO-A-2004/001664 (U.S. Ser. No. 10/518,421) supra. The loading member 161 would be sized and shaped to fit over, and around, the dose counter, as will be better understood with reference to the embodiment described with reference to FIGS. 6 to 11 which utilises such a dose counter.

FIGS. 6 to 11 illustrate a hand-held, hand-operable inhaler of the pMDI type in accordance with a third embodiment of the present invention. The pMDI of this embodiment is very similar to the embodiment previously described with reference to FIGS. 1 to 3, and therefore only the differences will be described in any detail, with the like parts being assigned like reference numerals.

A first difference is that the canister 205 has a dose counter 285 mounted on its head; i.e. on the end of the canister 205 at which the metering valve is disposed. The dose counter 285 in this embodiment is as described and shown in WO-A-2004/001664 (U.S. Ser. No. 10/518,421) supra, incorporated herein by reference. Moreover, the dose counter 285 is permanently fixed to the can head with a split-ring collar as described and shown in US-A-2003/0136800 or WO-A-2004/065224 (U.S. Ser. No. 10/543,049), incorporated herein by reference. The canister 205 and dose counter 285 thus form a unit.

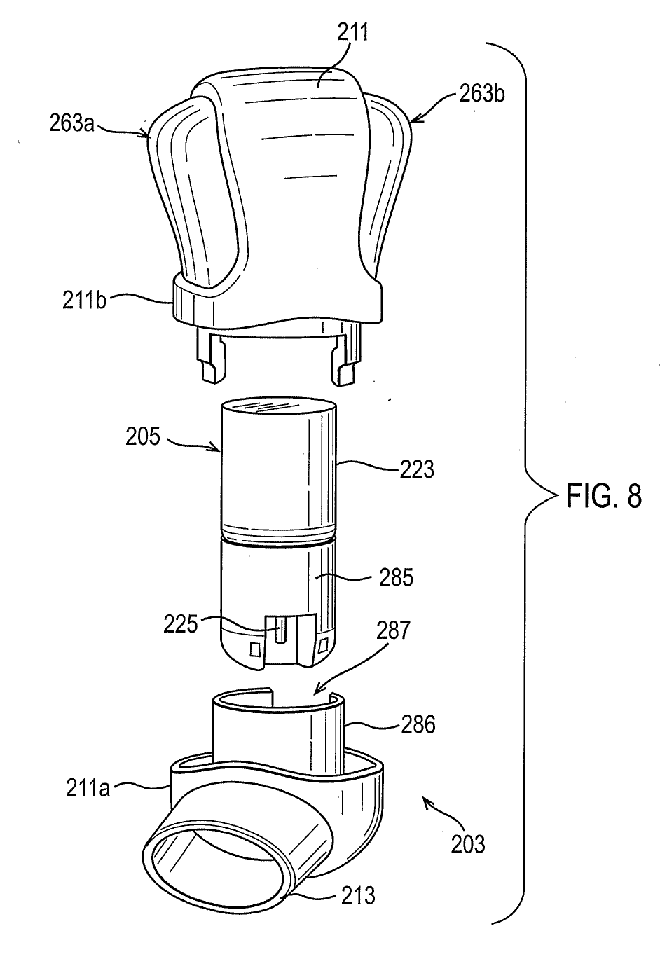

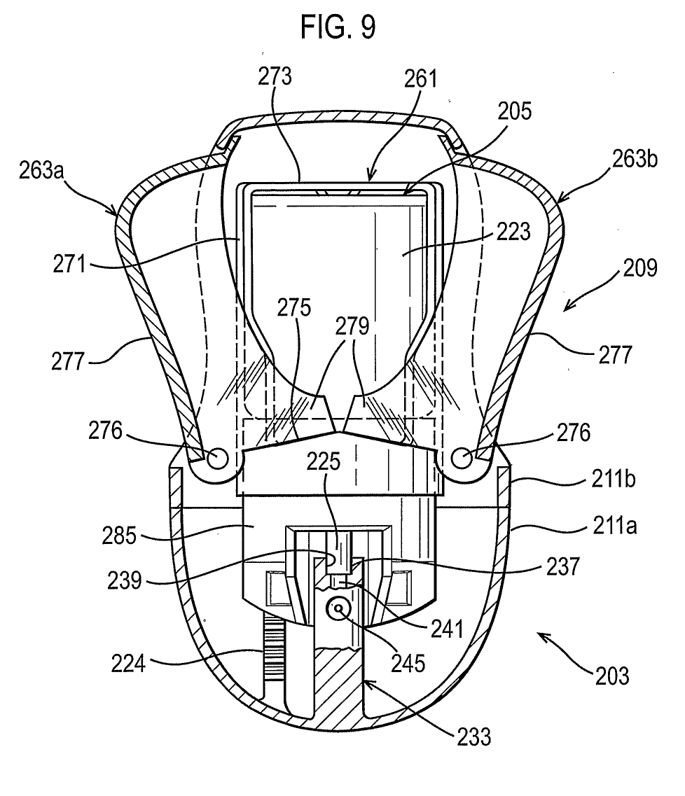

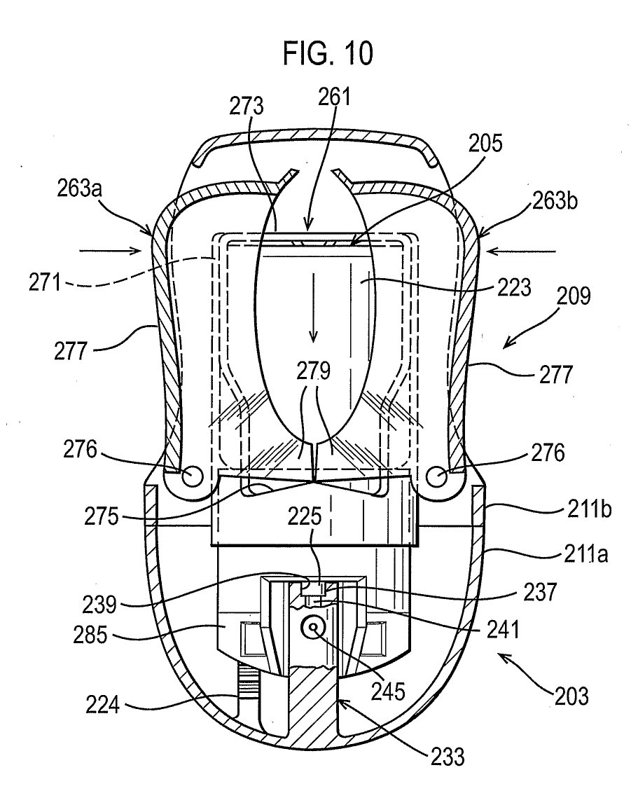

The dose counter 285 has a display (not shown) on the rear side as viewed in FIG. 8. The housing 211 is provided with a window or aperture (not shown) at a position to register with the display when the canister-counter unit 205, 285 is mounted in the actuator, whereby the patient is able to see the dose counter display. As shown in FIGS. 9 and 10, disposed on the base surface of the housing 211, beside the nozzle block 233, is a rack 224 for driving the dose counter 285 when the canister 205 is actuated by the actuating mechanism 209, as will be understood by reference to WO-A-2004/001664 (U.S. Ser. No. 10/518,421) supra.

In this embodiment the housing 211 is split into a lower housing part 211a and an upper housing part 211b which are assemblable by a bayonet fitting, more details of which follow hereinafter.

The lower housing part 211a comprises the mouthpiece 213 and the nozzle block 233. The lower housing part 211a further has an upstanding skirt 286 in which is formed a slot 287. The slot 287 receives that part of the dose counter 285 having the display and cooperates with the dose counter display part to ensure alignment of the display with the aforementioned window in the housing 211.

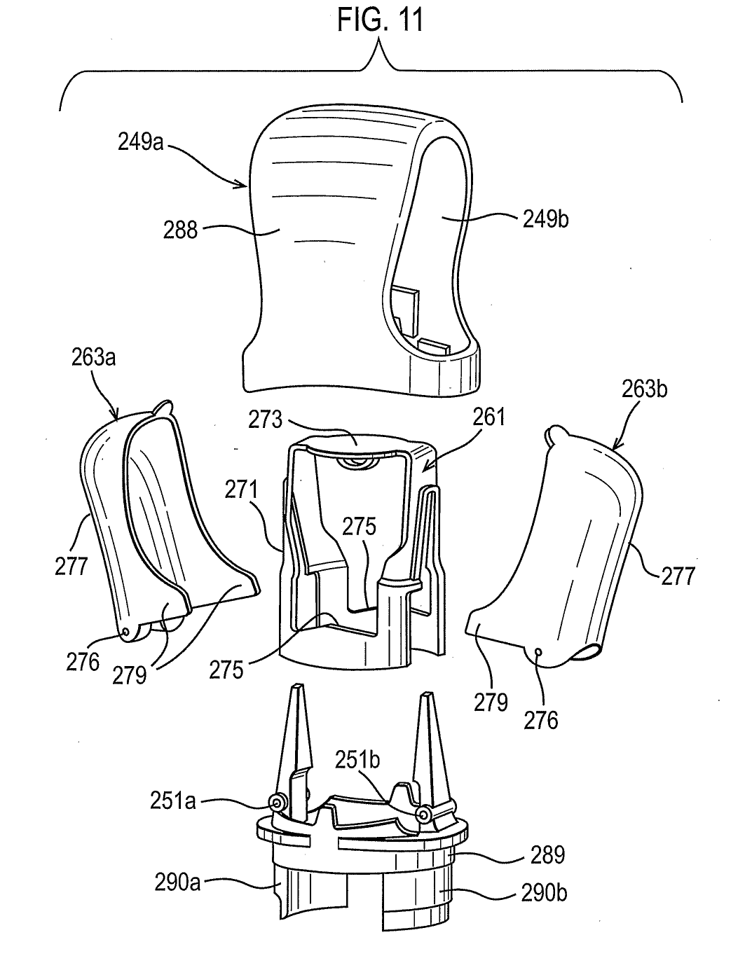

Referring to FIG. 11, the upper housing part 211b comprises a hollow, shell-like upper body 288, of generally inverted T-shape, which presents the lateral apertures 249a, b for receipt of the actuating members 263a, b. The upper housing part 211b further comprises a chassis 289 which provides the pivot elements 251a, 251b for engagement with the pivot elements 276 of the actuating members 263a, b for pivotal movement thereof in the lateral apertures 249a, b between the rest and actuated positions shown in FIGS. 9 and 10, respectively.

The chassis 289 includes a pair of arms 290a, b at its lower end which form the male part of a bayonet fitting which connects the lower and upper housing parts 211a, b to form the housing 211. The female part of the bayonet fitting is provided in the lower housing part 211a (not shown). The operation of the bayonet fitting will be understood by reference to FIGS. 6 to 8.

As shown particularly clearly in FIG. 11, the loading member 261 in this embodiment is of a different shape and configuration than in the previous embodiments. FIG. 11 also shows that the actuating members 263a, 263b are hollow, shell-like members, each presenting a pair of loading arms 279 which, in use, straddle the canister 205 to act on the loading section 275 on opposing sides of the canister 205.

As will be understood by reference to FIGS. 9 and 10, operation of the inhaler is the same as for the inhaler illustrated in FIGS. 1 to 3. However, when the actuator is actuated through inward pivotal movement of the actuating members 263a, 263b, the resulting downward movement of the canister-counter unit 205, 285 in the housing 211 not only results in the metering valve being opened, for dispensement of a metered dose of medicament, but also the stationary rack 224 riding up in an aperture (not shown) in the dose counter 285 for advancement of the display thereof, as detailed in WO-A-2004/001664 (U.S. Ser. No. 10/518,421) supra.

FIG. 12 shows a modification to the upper part of the actuator of the inhaler of FIGS. 6 to 11 in which the actuating members 263a, b are integrally formed with the chassis 289, e.g. as a one-piece moulding. The actuating member 263a, b are linked to the chassis 289 through living hinges 291 to provide the required pivotal movement of the actuating members 263a, b.

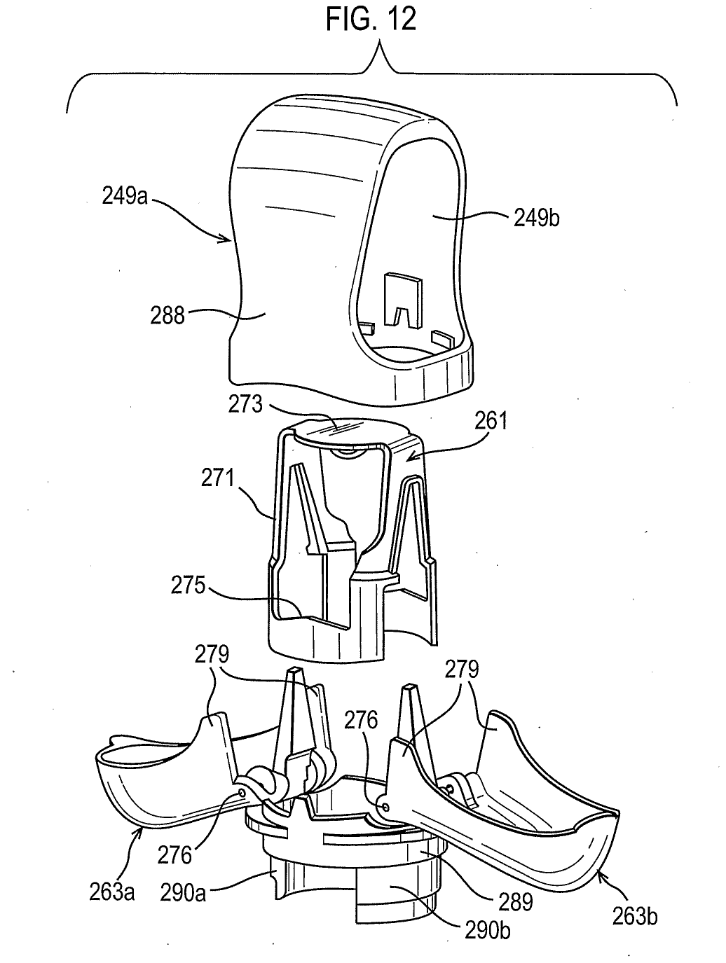

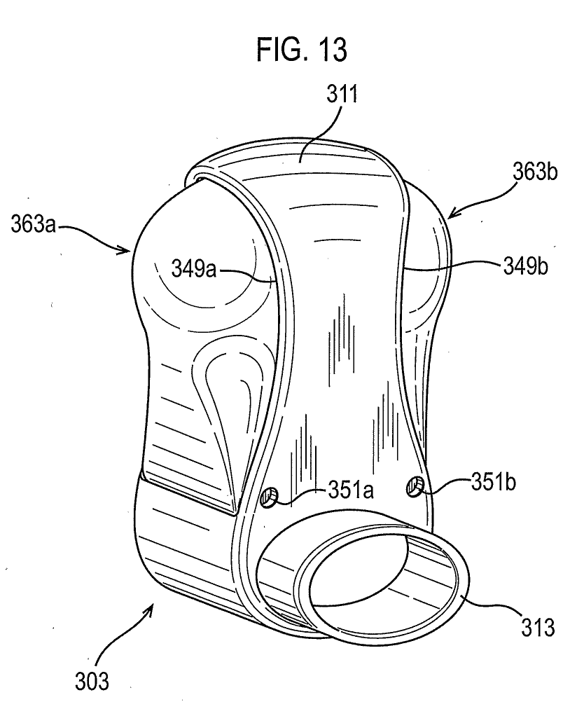

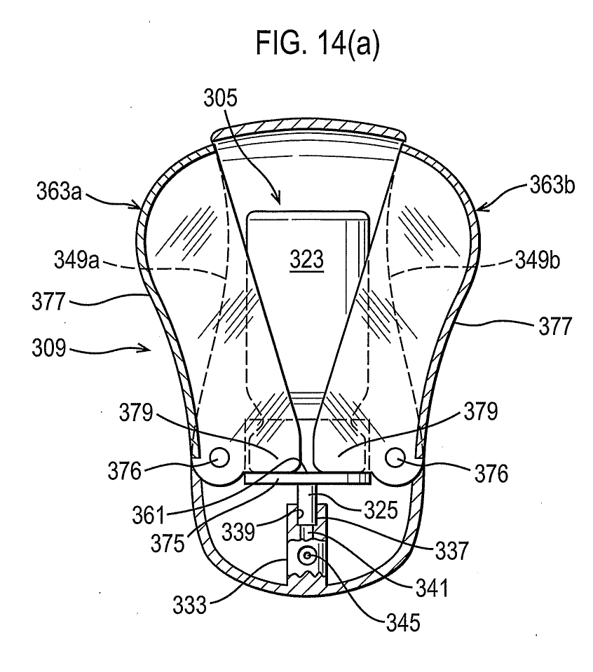

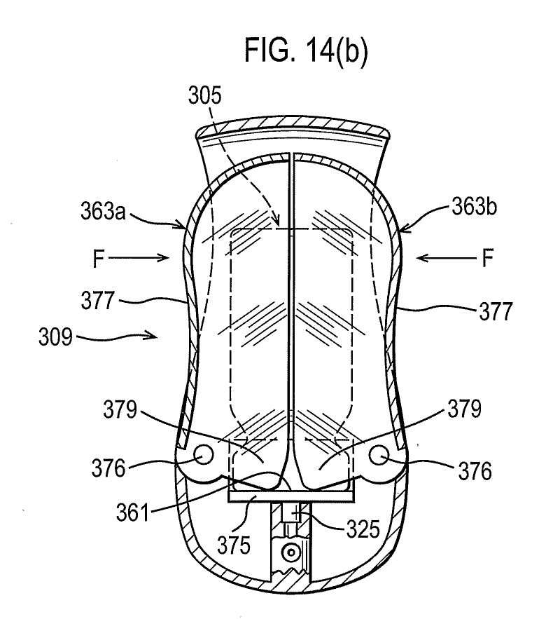

FIGS. 13 to 15 illustrate a hand-held, hand-operable inhaler of the pMDI type in accordance with a fourth embodiment of the present invention, noting that there are some styling differences between the representations in FIGS. 13 and 14. The inhaler of this embodiment is similar in respects to the inhaler of the first embodiment shown in FIGS. 1 to 3, and thus, in order to avoid unnecessary duplication of description, only the differences will be described in detail, with like parts being designated by like reference signs.

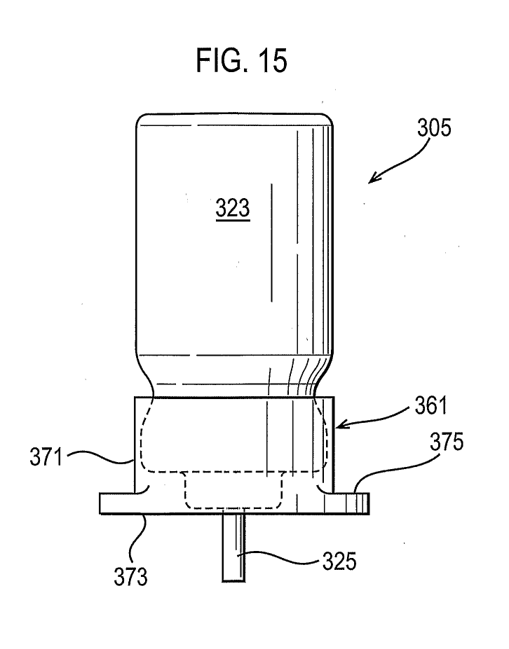

The actuation mechanism 309 in this embodiment comprises a loading member 361 which, as shown clearly in FIG. 15, is fitted over the head of the body 323 of the canister 305. In this embodiment the loading member 361 is configured as a cap member. Preferably, the loading member 361 is permanently fitted to the head of the body 323 of the canister 305, for instance through use of a split-ring collar as described in US-A-2003/0136800 or WO-A-2004/065224 (U.S. Ser. No. 10/543,049) supra. However, non-permanent fits could be used, for instance a snap fit connection.

The loading member 361 is slidably disposed relative to the nozzle block 333 between a first, rest or inoperative position, as illustrated in FIG. 14(a), and a second, actuated position in which the canister 305 is actuated, as illustrated in FIG. 14(b), and comprises a sleeve 371, here a tubular sleeve, which is a close fit with the outer peripheral wall of the head of the canister 305, an end section 373 at one, the lower, end of the sleeve 371, here which spans the sleeve 371, which engages the head of the body 323 of the canister 305, and which presents an annular loading section 375 which is engaged by the actuating members 363a, b to load the canister 305. Of course, the loading section 375 need not necessarily be annular, but instead provided as first and second lateral flanges for the actuating members 363a, b to act on.

Operation of the actuator is as for the first embodiment of FIGS. 1 to 3. In other words, inward rotation of the actuating members 363a, b causes the loading arms 379 of the actuating members 363a, b to drive the loading section 375 of the loading member 361, and hence the loading member 361, downwardly, which downward movement of the loading member 361 drives or pulls the canister body 323 of the canister 305 downwardly in relation to the valve stem 325 of the canister 305 which is held stationary by the nozzle block 333.

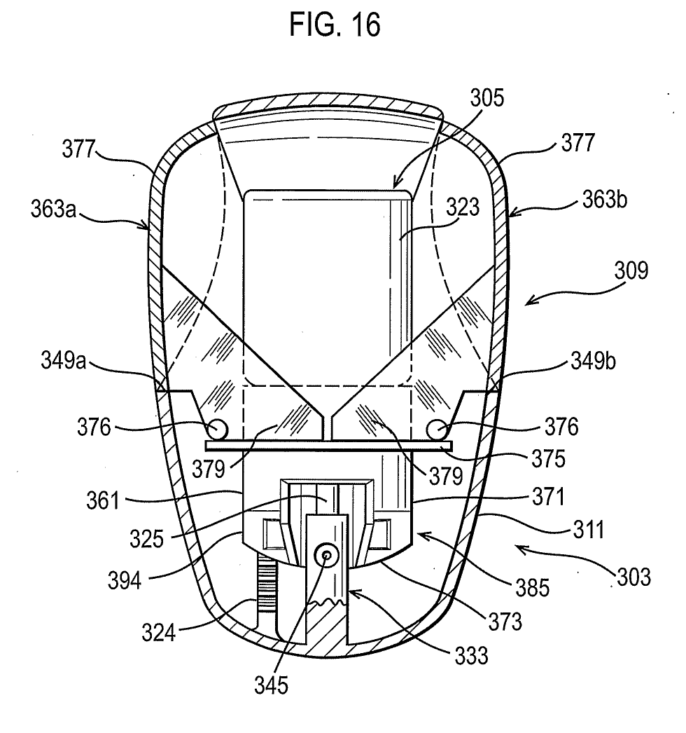

FIGS. 16 to 18 illustrate a hand-held, hand-operable inhaler of the pMDI type in accordance with a fifth embodiment of the present invention. The inhaler of this embodiment is very similar to the inhaler of the above-described fourth embodiment, and thus, in order to avoid unnecessary duplication of description, only the differences will be described in detail, with like parts being designated by like reference signs.

The inhaler of this embodiment differs from that of the fourth embodiment in the construction of the loading member 361. In this embodiment, the loading member 361 is provided as a modification of the dose counter 385 described in WO-A-2004/001664 (U.S. Ser. No. 10/518,421) supra, which patent applications are incorporated herein by reference.

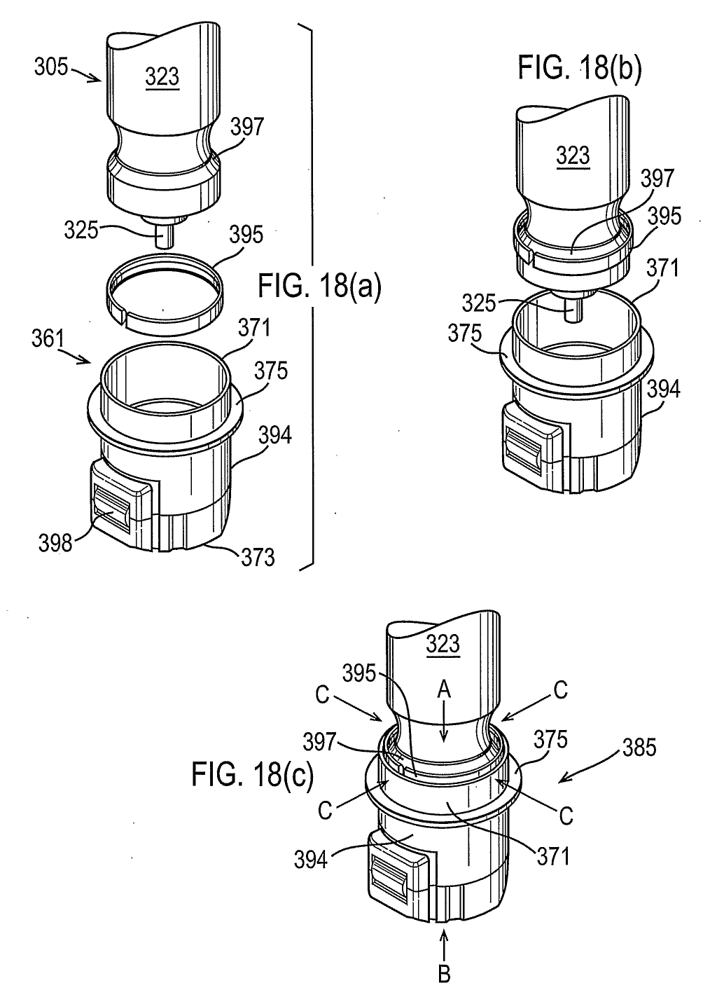

The dose counter 385 is permanently fixed to the head of the canister body 323, to form a unit therewith, through use of a split ring collar 395 detailed in US-A-2003/0136800 or WO-A-2004/065224 (U.S. Ser. No. 10/543,049), all of which are incorporated herein by reference, and as will now be briefly described with reference to FIGS. 18(a)-(c).

FIGS. 18(a)-(c) illustrate the process whereby the collar 395 is fitted around the neck 397 of the canister body 323 and welded to the tubular sleeve 371 of the dose counter housing 394. FIG. 18(a) is an exploded diagram showing the collar 395 being positioned between the canister body 323 and the dose counter housing 394, which is here shown with a counter window 398 in which is a display (not shown) of the number of metered doses of the medicament which are left in the canister 305, or which have been dispensed from the canister 305. FIG. 18(b) shows the collar 395 having been slipped around the neck 397 by opening the collar 395, sliding it over the head of the canister body 323 and then allowing the return force in the collar 395 to close it onto the neck 397. As shown in FIG. 18(c), the collar 395 is slid over the canister 305 in the direction of arrow A thereby causing the collar 395 to radially expand due to the interaction of the inner circumferential surface of the collar 395 with the flaring surface of the neck 397. Meanwhile, the dose counter housing 394 is positioned over the head of the canister body 323 by being moved down in the direction of arrow B. In this way, an inner end wall (not shown) of the housing 394 abuts the head of the canister body 323 and the collar 395 is wedged between the inner surface of the sleeve 371 and the neck 397. The collar 395 is then joined to the inner surface of the sleeve 371 by ultrasonic welding at the points indicated by arrows C, thereby permanently securing the dose counter 385 to the canister body 323.

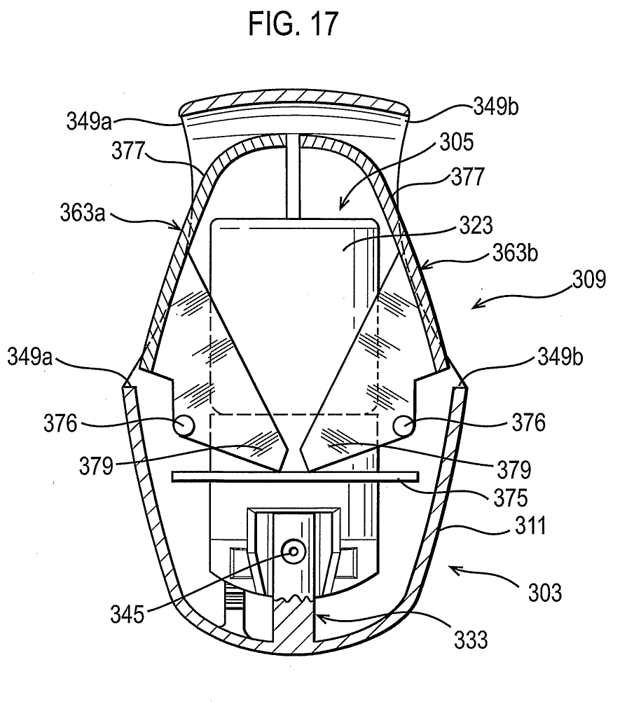

As shown in FIGS. 16 and 17, operation of the inhaler of this fifth embodiment is the same as for the above-described fourth embodiment, with the loading section 375 on which the loading arms 379 act being provided as an annular flange or projection on the dose counter housing 394.

FIGS. 16 and 17 show the rack 324 disposed to the base surface of the housing 311, beside the nozzle block 333, which drives the dose counter 385 when the canister 305 is actuated by the actuating mechanism 309, as described in WO-A-2004/001664 (U.S. Ser. No. 10/518,421) supra, incorporated herein by reference.

The display in the dose counter window 398 is updated upon each actuation event and the display is visible to the patient or user through a window or aperture provided in the actuator housing 311 (not shown) at a position to register with the display when the canister-counter unit 305, 385 is mounted in the actuator housing 311.

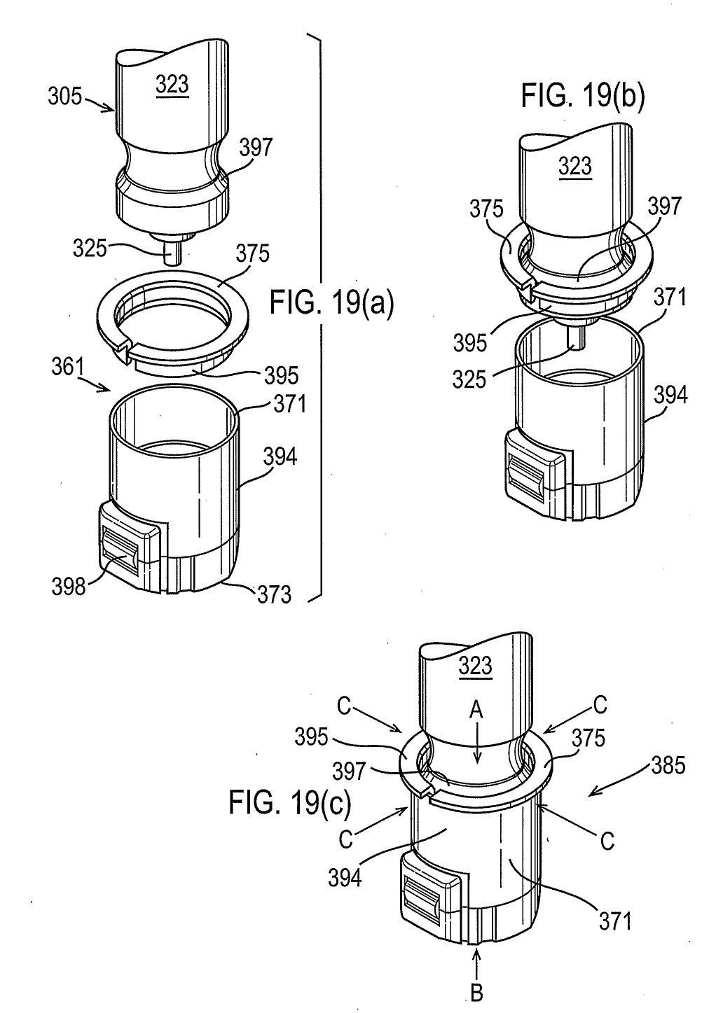

FIGS. 19(a)-(c) show a modification to the fifth embodiment where the loading section 375 is provided by the split-ring collar 395 instead of the dose counter housing 394. Operation of the inhaler is otherwise the same as described with reference to FIGS. 16 to 18.

As will be appreciated, the loading member 361 may take the form of other accessories which are fixedly connected to the head end of the canister body 323.

In an alternative embodiment of the present invention, not shown, the loading section for the actuating members to act on may be presented by a surface of the canister.

It will be appreciated that the actuating mechanisms in the illustrated embodiments of the invention provide a mechanical advantage. That is to say, the manual force required to be applied by the user to operate the inhaler (by overcoming the return force of the valve return spring) is less than would otherwise be the case, such as in operation of a standard pMDI where the user has to push down on the base of the canister against the return force of the valve return spring.

Preferably, all of the parts of the actuator of the exemplary embodiments are made from a plastics material, for example by a moulding process.

The medicament contained in the aerosol canister may for the treatment of mild, moderate or severe acute or chronic symptoms or for prophylactic treatment. The medicament is suitably for treating respiratory diseases, e.g. asthma, chronic obstructive pulmonary disease (COPD), although may be for other therapeutic indications, e.g. treating rhinitis.

Appropriate therapeutic agents or medicaments may thus be selected from, for example, analgesics, e.g., codeine, dihydromorphine, ergotamine, fentanyl or morphine; anginal preparations, e.g., diltiazem; antiallergics, e.g., cromoglycate (e.g. as the sodium salt), ketotifen or nedocromil (e.g. as the sodium salt); antiinfectives e.g., cephalosporins, penicillins, streptomycin, sulphonamides, tetracyclines and pentamidine; antihistamines, e.g., methapyrilene; anti-inflammatories, e.g., beclomethasone (e.g. as the dipropionate ester), fluticasone (e.g. as the propionate ester), flunisolide, budesonide, rofleponide, mometasone (e.g. as the furoate ester), ciclesonide, triamcinolone (e.g. as the acetonide), 6α, 9α-difluoro-11β-hydroxy-16α-methyl-3-oxo-17α-propionyloxy-androsta-1,4-diene-17β-carbothioic acid S-(2-oxo-tetrahydro-furan-3-yl) ester or 6α,9α-Difluoro-17α-[(2-furanylcarbonyl)oxy]-11β-hydroxy-16α-methyl-3-oxo-androsta-1,4-diene-17β-carbothioic acid S-fluoromethyl ester; antitussives, e.g., noscapine; bronchodilators, e.g., albuterol (e.g. as free base or sulphate), salmeterol (e.g. as xinafoate), ephedrine, adrenaline, fenoterol (e.g. as hydrobromide), formoterol (e.g. as fumarate), isoprenaline, metaproterenol, phenylephrine, phenylpropanolamine, pirbuterol (e.g. as acetate), reproterol (e.g. as hydrochloride), rimiterol, terbutaline (e.g. as sulphate), isoetharine, tulobuterol or 4-hydroxy-7-[2-[[2-[[3-(2-phenylethoxy)propyl]sulfonyl]ethyl]amino]ethyl-2(3H)benzo-thiazolone; PDE4 inhibitors e.g. cilomilast or roflumilast; leukotriene antagonists e.g. montelukast, pranlukast and zafirlukast; [adenosine 2a agonists, e.g. 2R,3R,4S,5R)-2-[6-Amino-2-(1S-hydroxymethyl-2-phenyl-ethylamino)-purin-9-yl]-5-(2-ethyl-2H-tetrazol-5-yl)-tetrahydro-furan-3,4-diol (e.g. as maleate)]; [α4 integrin inhibitors e.g. (2S)-3-[4-({[4-(aminocarbonyl)-1-piperidinyl] carbonyl}oxy)phenyl]-2-[((2S)-4-methyl-2-{[2-(2-ethylphenoxy)acetyl]amino}pentanoyl)amino] propanoic acid (e.g as free acid or potassium salt)], diuretics, e.g., amiloride; anticholinergics, e.g., ipratropium (e.g. as bromide), tiotropium, atropine or oxitropium; hormones, e.g., cortisone, hydrocortisone or prednisolone; xanthines, e.g., aminophylline, choline theophyllinate, lysine theophyllinate or theophylline; therapeutic proteins and peptides, e.g., insulin or glucagons. It will be clear to a person skilled in the art that, where appropriate, the medicaments may be used in the form of salts, (e.g., as alkali metal or amine salts or as acid addition salts) or as esters (e.g., lower alkyl esters) or as solvates (e.g., hydrates) to optimise the activity and/or stability of the medicament and/or to minimise the solubility of the medicament in the propellant.

Preferably, the medicament is an anti-inflammatory compound for the treatment of inflammatory disorders or diseases such as asthma and rhinitis.

Preferably, the medicament is formulated in a hydrofluoroalkane propellant, such as HFA-134a or HFA-227 or a combination thereof.

Preferably, the medicament is an anti-inflammatory steroid, such as a corticosteroid, for instance fluticasone, e.g. as the propionate ester, or a long acting beta agonist (LABA), such as salmeterol, e.g. as the xinafoate salt, or a combination thereof.

Preferred medicaments are salmeterol, salbutamol, albuterol, fluticasone and beclomethasone and salts, esters or solvates thereof, for instance fluticasone propionate, albuterol sulphate, salmeterol xinafoate and beclomethasone diproprionate.

The medicament may also be a glucocorticoid compound, which has anti-inflammatory properties. One suitable glucocorticoid compound has the chemical name: 6α,9α-Difluoro-17α-(1-oxopropoxy)-11β-hydroxy-16α-methyl-3-oxo-androsta-1,4-diene-17β-carbothioic acid S-fluoromethyl ester (fluticasone propionate). Another suitable glucocorticoid compound has the chemical name: 6α,9α-difluoro-17α-[(2-furanylcarbonyl)oxy]-11β-hydroxy-16α-methyl-3-oxo-androsta-1,4-diene-17β-carbothioic acid S-fluoromethyl ester. A further suitable glucocorticoid compound has the chemical name: 6α,9α-Difluoro-11β-hydroxy-16α-methyl-17α-[(4-methyl-1,3-thiazole-5-carbonyl)oxy]-3-oxo-androsta-1,4-diene-17β-carbothioic acid S-fluoromethyl ester.

Other suitable anti-inflammatory compounds include NSAIDs e.g. PDE4 inhibitors, leukotriene antagonists, iNOS inhibitors, tryptase and elastase inhibitors, beta-2 integrin antagonists and adenosine 2a agonists.

The medicaments may be delivered in combinations. As an example, there may be provided salbutamol (e.g. as the free base of the sulphate salt) or salmeterol (e.g. as the xinafoate salt) in combination with an anti-inflammatory steroid, such as beclomethasone (e.g. as an ester, preferably dipropionate) or fluticasone (e.g. as an ester, preferably propionate).

Finally, it will be understood that the present invention has been described in its exemplary embodiments and can be modified in many different ways without departing from the scope of the invention as defined by the appended claims.

Also, as regards the provision of reference signs in the appended claims, it is to be understood that reference signs are provided only for illustrative purposes and are not intended to confer any limitation to the claimed invention.

What is claimed is:

1. An inhaler for delivering medicament by inhalation, comprising:
a canister which comprises a body which includes a base and a head and defines a chamber containing medicament, and a valve stem which extends from the body and from which medicament is in use delivered on actuation of the canister; and
an actuator comprising a main body comprising a housing receiving the canister, and an actuation mechanism for actuating the canister;
wherein the actuation mechanism comprises a loading member which is fitted to or comprised in the canister and includes a loading section which is located at a distance spaced from the base of the body of the canister and, in use, acted upon to drive the loading member in an actuating direction from a first, rest position to a second, actuated position in which the canister is actuated to deliver medicament, and at least one actuating member which is actuatable by a user to drive the loading member in the actuating direction to the actuated position, such as to actuate the canister to deliver medicament;
wherein the at least one actuating member is pivotally coupled to the housing for pivotal movement relative to the housing from a first, rest position to a second, actuated position in which the loading member is driven in the actuating direction to the actuated position, such as to actuate the canister to deliver medicament; and
wherein the at least one actuating member comprises a gripping element which extends along a length of the housing and is configured to be gripped and depressed by the user in actuating the canister.

2. The inhaler of claim 1, wherein the loading member is located over the base of the body of the canister.

3. The inhaler of claim 2, wherein the loading member comprises a sleeve which fits about an outer peripheral surface of the body of the canister, an end section at one end of the sleeve which engages the base of the body of the canister, and the loading section at the other end of the sleeve.

4. The inhaler of claim 3, wherein the sleeve of the loading member extends substantially to the head of the body of the canister.

5. The inhaler of claim 3, wherein the loading section of the loading member comprises a substantially annular section.

6. The inhaler of claim 1, wherein the loading member is attached substantially to the head of the body of the canister.

7. The inhaler of claim 6, wherein the loading section of the loading member comprises a substantially annular section.

8. The inhaler of claim 1, wherein the at least one actuating member comprises a loading element for engaging the loading section of the loading member when the at least one actuating member pivots from the first, rest position to the second, actuated position to drive the loading member in the actuating direction to the actuated position, such as to actuate the canister to deliver medicament.

9. The inhaler of claim 8, wherein the gripping element extends from a pivot along a length of the housing and the loading element extends inwardly from the pivot (176; . . . ).

10. The inhaler of claim 1, wherein the housing includes at least one lateral opening in which the at least one actuating member is disposed for depression by the user.

11. The inhaler of claim 1, wherein the actuation mechanism comprises first and second actuating members which are disposed in oppositely-directed relation.

12. The inhaler of claim 1, wherein the actuation mechanism further comprises at least one toggle element which acts normally to bias the loading member in a direction opposite the actuating direction and is configured to toggle between a first, restraining configuration and a second, actuated configuration on the application of a predeterminable actuation force to the loading member in the actuating direction.

13. The inhaler of claim 12, wherein the at least one toggle element comprises an elongate resilient element which extends substantially orthogonally to the actuating direction and engages the loading member such as normally to adopt a first bowed configuration which biases the loading member in a direction opposite the actuating direction and, on application of the actuation force, is toggled to a second, oppositely bowed configuration, which allows for actuation of the canister.

14. The inhaler of claim 13, wherein the second bowed configuration is not a stable state, such that, on release of the actuation force, the at least one toggle element returns to the first bowed configuration, thereby causing the loading member to return to the rest configuration.

15. The inhaler of claim 12, wherein the actuation mechanism comprises first and second toggle elements which are disposed to opposed sides of the canister.

16. The inhaler of claim 12, wherein the main body includes a nozzle block which receives the valve stem of the canister.

17. The inhaler of claim 16, wherein the housing includes an outlet member through which the user in use inhales.

18. The inhaler of claim 17, wherein the outlet member is a mouthpiece.

19. The inhaler of claim 1, wherein the actuation mechanism comprises a commitment feature which is configured and arranged such that the at least one actuating member is only able to pivot from the first, rest position to the second, actuated position when the user applies to the gripping element a force which is at least equal to a predetermined threshold force.

20. The inhaler of claim 4, wherein the loading section of the loading member comprises a substantially annular section.

21. The inhaler of claim 11 wherein the actuation mechanism further comprises at least one toggle element which acts normally to bias the loading member in a direction opposite the actuating direction and is configured to toggle between a first, restraining configuration and a second, actuated configuration on the application of a predeterminable actuation force to the loading member in the actuating direction.

22. An inhaler of claim 14, wherein the actuation mechanism comprises first and second toggle elements which are disposed to opposed sides of the canister.

23. The inhaler of claim 11, wherein the actuation mechanism comprises a commitment feature which is configured and arranged such that the at least one actuating member is only able to pivot from the first, rest position to the second, actuated position when the user applies to the gripping element a force which is at least equal to a predetermined threshold force.
